(19) United States
(12) Patent Application Publication
(10) Pub. No.: US 2024/0374596 A1
(43) Pub. Date: Nov. 14, 2024

(54) COMMIPHORA MYRRHA RESIN EXTRACT WITH NON-COVALENTLY ASSOCIATED VITAMIN B1 AND VITAMIN C COMPOUNDS

(71) Applicant: KING FAISAL UNIVERSITY, AL-AHSA (SA)

(72) Inventors: IMENE BAYACH, Al-Ahsa (SA); AMEL IBRAHIM, Al-Ahsa (SA)

(21) Appl. No.: 18/195,921

(22) Filed: May 10, 2023

(51) Int. Cl. A61K 31/51; A61K 31/375; A61K 36/328

(52) U.S. Cl. CPC A61K 31/51; A61K 31/375; A61K 36/328; A61K 2236/15; A61K 2236/17; A61K 2236/53

(57) ABSTRACT

A composition containing a Commiphora myrrha Resin extract, particularly a Commiphora myrrha Resin extract having non-covalently associated vitamin B1 and vitamin C compounds. Vitamin C derivatives can be added to the extract to promote synergistic antioxidant effects between vitamin B1 compounds and vitamin C compounds.

### BACKGROUND

### 1. Field

The disclosure of the present patent application relates to a Commiphora myrrha Resin extract, and particularly to a Commiphora myrrha Resin extract having non-covalently associated vitamin B1 and vitamin C compounds.

### 2. Description of the Related Art

It is well known that an average person requires for complete health a certain minimum of mineral and vitamin substances, all of which occur naturally in various foods. The most important among these substances are the minerals, such as calcium, phosphorus and iron, and the vitamin substances, such as vitamin A, vitamin B complex (thiamin or its hydrochloride, riboflavin, nicotinic acid or nicotinamide; pyridoxine, pantothenic acid, choline, inositol p-aminobenzoic acid, biotin, chondroitin and other chemically unidentified factors), vitamin C (ascorbic acid) and vitamin D. Other vitamin substances include vitamin E (alpha-tocopherol), vitamin K and others of less significance from a dietary point of view. In general, the vitamins can be divided into two groups: (1) the oil-soluble vitamins which include A, D, E and K among the more common vitamins, and (2) the water-soluble vitamins which include the remainder of those identified above.

Although it is possible to acquire the necessary amounts of all of these materials in a well-regulated diet, it is well established that a relatively large number of people do not receive amounts which are regarded as the minimum for complete health. For this reason, dietary deficiencies frequently occur.

Much progress has been made in alleviating dietary deficiencies based on the isolation, concentration, or both, of the vitamin substances which now are readily available commercially, alone or in various admixtures. As a result, diets are frequently supplemented with one or more vitamin substances, often without regard to the proper balance between them.

Many of the vitamin substances, both as they occur naturally and in pure or concentrated commercial forms, are unstable and the preparation of these materials in a stable form for ordinary uses has been subject to much difficulty. For example, many of the vitamins are deleteriously affected by the oxygen of the air, moisture, or both, by acids or alkaline substances, by other vitamins, or by minerals.

Vitamins B1 and C are major natural antioxidants capable of preventing damage generated by oxidative stress. Accordingly, there remains a need to provide stable compositions containing both vitamin B1 and vitamin C compounds to serve as antioxidants.

### SUMMARY

The presently disclosed subject matter relates to compositions obtained from Commiphora myrrha Resin containing one or more vitamin B1 compounds and one or more vitamin C compounds having a non-covalent association formed therebetween. Antioxidant activity is significantly enhanced in these complexes.

Accordingly, in one embodiment, the present subject matter relates to a composition comprising: one or more vitamin B1 compounds; and one or more vitamin C compounds selected from the group consisting of vitamin C and vitamin C derivatives, wherein the vitamin C derivatives have the formula:

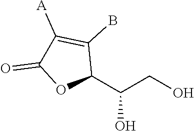

wherein each of A and B are independently OH, F, or methoxy, with the proviso that A and B are different from one another; and wherein there is a non-covalent association between the one or more vitamin B1 compounds and the one or more vitamin C compounds.

In another embodiment, the presently disclosed subject matter relates to a method of treating an oxidant condition in a subject, comprising administering a therapeutically effective amount of the compositions as described herein to a subject in need thereof.

In a further embodiment, the present subject matter relates to a method of producing an antioxidant composition, the method comprising: drying a sample of Commiphora myrrha Resin to obtain a dried resin; grinding the dried resin to obtain a fine powder; extracting a vitamin sample from the fine powder using Soxhlet extraction to obtain an extract; filtering the extract to obtain a filtered composition; and adding one or more vitamin C derivatives to the filtered composition to obtain the antioxidant composition containing one or more vitamin B1 compounds and one or more vitamin C compounds selected from the group consisting of vitamin C and the vitamin C derivatives, wherein the one or more vitamin C derivatives have the formula:

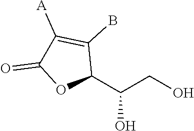

wherein each of A and B are independently OH, F, or methoxy, with the proviso that A and B are different from one another; and wherein there is a non-covalent association between the one or more vitamin B1 compounds and the one or more vitamin C compounds.

These and other features of the present subject matter will become readily apparent upon further review of the following specification.

### BRIEF DESCRIPTION OF THE DRAWINGS

FIGS. 1A-1C show 3d structures of the non-covalent complexes with vitamin B1 and vitamin C (ascorbic acid) derivatives described herein, with the following vitamin C derivatives being shown in the FIGURES: 1A) 2F-ascorbic acid, 1B) 3F-ascorbic acid, and IC) 3-methyl-ascorbic acid.

### DETAILED DESCRIPTION OF THE PREFERRED EMBODIMENTS

The following definitions are provided for the purpose of understanding the present subject matter and for construing the appended patent claims.

### Definitions

Throughout the application, where compositions are described as having, including, or comprising specific components, or where processes are described as having, including, or comprising specific process steps, it is contemplated that compositions of the present teachings can also consist essentially of, or consist of, the recited components, and that the processes of the present teachings can also consist essentially of, or consist of, the recited process steps.

It is noted that, as used in this specification and the appended claims, the singular forms “a”, “an”, and “the” include plural references unless the context clearly dictates otherwise.

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition or a method described herein can be combined in a variety of ways without departing from the spirit and scope of the present teachings, whether explicit or implicit herein.

The use of the terms “include,” “includes”, “including,” “have,” “has,” or “having” should be generally understood as open-ended and non-limiting unless specifically stated otherwise.

“Subject” as used herein refers to any animal classified as a mammal, including humans, domestic and farm animals, and zoo, sports, and pet companion animals such as household pets and other domesticated animals such as, but not limited to, cattle, sheep, ferrets, swine, horses, poultry, rabbits, goats, dogs, cats and the like.

“Patient” as used herein refers to a subject in need of treatment of a condition, disorder, or disease, such as an oxidant condition.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10% variation from the nominal value unless otherwise indicated or inferred.

The term “optional” or “optionally” means that the subsequently described event or circumstance may or may not occur, and that the description includes instances where said event or circumstance occurs and instances in which it does not.

Unless defined otherwise, all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

Throughout the application, descriptions of various embodiments use “comprising” language. However, it will be understood by one of skill in the art, that in some specific instances, an embodiment can alternatively be described using the language “consisting essentially of” or “consisting of”.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

The presently disclosed subject matter relates to compositions obtained from Commiphora myrrha Resin containing one or more vitamin B1 compounds and one or more vitamin C compounds having a non-covalent association formed therebetween. Antioxidant activity is significantly enhanced in these complexes.

Accordingly, in one embodiment, the present subject matter relates to a composition comprising: one or more vitamin B1 compounds; and one or more vitamin C compounds selected from the group consisting of vitamin C and vitamin C derivatives, wherein the vitamin C derivatives have the formula:

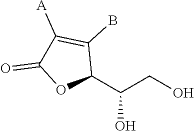

wherein each of A and B are independently OH, F, or methoxy, with the proviso that A and B are different from one another; and wherein there is a non-covalent association between the one or more vitamin B1 compounds and the one or more vitamin C compounds.

In an embodiment, the non-covalent association between the one or more vitamin B1 compounds and the one or more vitamin C compounds is hydrogen bonding. In any event, the non-covalent association can be formed between the one or more vitamin B1 compounds and the vitamin C and/or the vitamin C derivatives. In another embodiment, the one or more vitamin B1 compounds and the vitamin C can be extracted from Commiphora myrrha resin. The Commiphora myrrha resin extract as obtained herein can have the non-covalent association between the one or more vitamin B1 compounds and the vitamin C. Upon addition of the one or more vitamin C derivatives, similar non-covalent associations will form between the one or more vitamin B1 compounds and the one or more vitamin C derivatives.

In an embodiment, the one or more vitamin B1 compounds can have the formula:

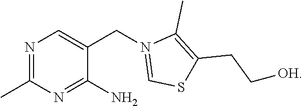

In another embodiment, the vitamin C can have the formula:

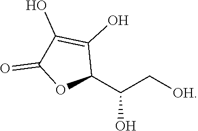 [SMILES: O=C1O[C@H]([C@@H](O)CO)C(O)=C1O]

In a further embodiment, the vitamin C derivatives can be one or more of the following compounds 1-4:

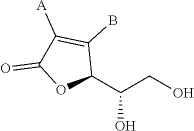

| | A | B |
|---|---|---|
| 1 | OH | F |
| 2 | F | OH |
| 3 | OH | Methoxy |
| 4 | Methoxy | OH |

In an embodiment, once the association is formed, the one or more vitamin B1 compounds and the one or more Vitamin C compounds can exhibit a synergistic antioxidant effect due to the non-covalent association. In this regard, such antioxidants are recognized to act as free radical scavengers by following different mechanisms, including the hydrogen atom transfer (HAT) mechanism according to the following equation:

ArOH+R·→ArO·+RH

In another embodiment, the presently disclosed subject matter relates to a method of treating an oxidant condition in a subject, comprising administering a therapeutically effective amount of the compositions as described herein to a subject in need thereof. Accordingly, the compositions described herein can provide antioxidant activity as well as potentially other therapeutic uses, for example, in the treatment and management of inflammatory conditions.

In an embodiment, the one or more vitamin B1 compounds and the one or more Vitamin C compounds in the composition, when used in the present methods, can exhibit a synergistic antioxidant effect due to the non-covalent association. In certain embodiments, the non-covalent association is hydrogen bonding. In certain other embodiments, the non-covalent association can enhance antioxidant activity of the vitamin C derivatives.

In another embodiment, the antioxidant activity of the present compositions can be confirmed by referring to bond dissociation enthalpies (BDEs) for the vitamin C (ascorbic acid) derivatives with F and OMe substitutions at positions 2 and 3, both as single molecules and within a non-covalent complex with vitamin B1 (thiamine). These positions were selected because they are known to be critical for the antioxidant activity of ascorbic acid. The BDEs are important indicators of antioxidant activity, as lower BDE values generally correspond to higher antioxidant potential.

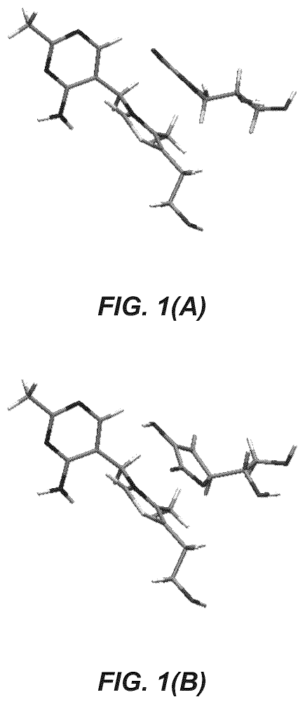

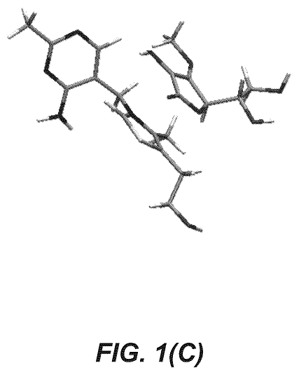

For example, the BDE values for the vitamin C derivatives within the non-covalent complex with vitamin B1 can be slightly lower than those of the corresponding single molecules for most derivatives. This suggests that the non-covalent association with vitamin B1 further enhances the antioxidant activity of these derivatives, as lower BDEs indicate higher antioxidant potential. The geometrical arrangement of the single molecules within the non-covalent complex of ascorbic acid derivatives with vitamin B1 also plays a crucial role in facilitating this synergistic effect and enhancing antioxidant activity (See FIGS. 1A-1C).

In this regard, the BDE of the molecules is defined as the enthalpy difference for the reaction, at 298 K and 1 atm:

MolOH→MolO·+H·

In some embodiments, the present compositions can be used for combination therapy, where other therapeutic and/or prophylactic ingredients can be co-administered therewith. The compositions described herein can further include a therapeutically acceptable carrier or excipient. Non-limiting examples of suitable excipients, carriers, or vehicles useful herein include liquids such as water, saline, glycerol, polyethylene glycol, hyaluronic acid, ethanol, and the like. Suitable excipients for nonliquid formulations are also known to those of skill in the art. In an embodiment, a therapeutically effective amount of the composition can include from about 0.01 mg to 500 mg per unit dose. For example, an effective amount of the composition can include from about 1 mg to about 100 mg per unit dose. The precise effective amount will vary from subject to subject and will depend upon the species, age, the subject's size and health, the nature and extent of the condition being treated, recommendations of the treating physician, and the therapeutics or combination of therapeutics selected for administration. The subject may be administered as many doses as is required to reduce and/or alleviate the signs, symptoms, or causes of the disease or disorder in question, or bring about any other desired alteration of a biological system.

The present compounds are typically administered at a therapeutically or pharmaceutically effective dosage, e.g., a dosage sufficient to provide treatment for an oxidant condition. Administration of the present compositions can be by any method that delivers the compositions systemically and/or locally. These methods include oral routes, parenteral routes, intraduodenal routes, and the like.

In employing the present compositions for treatment of oxidant conditions or other diseases, disorders, or conditions, any acceptable mode of administration can be used with other acceptable excipients, including solid, semi-solid, liquid or aerosol dosage forms, such as, for example, tablets, capsules, powders, liquids, suspensions, suppositories, aerosols, or the like. The present compositions can also be administered in immediate, sustained, or controlled release dosage forms, including depot injections, osmotic pumps, pills, transdermal (including electrotransport) patches, and the like, for the prolonged administration of the composition at a predetermined rate, preferably in unit dosage forms suitable for single administration of precise dosages.

The present compositions may also be administered as a “dietary supplement” or a product that is intended to supplement the human diet and may be provided in the form of a pill, capsule, tablet, or like formulation. By way of non-limiting example, a dietary supplement may include one or more of the following dietary ingredients: vitamins, minerals, herbs, botanicals, amino acids, and dietary substances intended to supplement the diet by increasing total dietary intake, or a concentrate, metabolite, constituent, extract, or combinations of these ingredients, not intended as a conventional food or as the sole item of a meal or diet. Dietary supplements may also be incorporated into foodstuffs, such as functional foods designed to promote control of glucose levels. A “functional food” is an ordinary food that has one or more components or ingredients incorporated into it to give a specific medical or physiological benefit, other than a purely nutritional effect.

In an embodiment, the composition may be in the form of an immediate-release, controlled-release, or sustained-release orally administrable composition for a pill, tablet, capsule, gelcap, lozenge, throat spray, solution, emulsion, cream, paste, gel, cough drop, or dissolvable strip. In an embodiment, the composition may be in the form of a liquid solution, liquid spray, emulsion, cream, gel, lotion, or impregnated dressing. In an embodiment, the composition may be in the form of nasal drops, oral drops, eye drops, or aerosol trigger.

In a further embodiment, the present subject matter relates to a method of producing an antioxidant composition, the method comprising: drying a sample of Commiphora myrrha Resin to obtain a dried resin; grinding the dried resin to obtain a fine powder; extracting a vitamin sample from the fine powder using Soxhlet extraction to obtain an extract; filtering the extract to obtain a filtered composition; and adding one or more vitamin C derivatives to the filtered composition to obtain the antioxidant composition containing one or more vitamin B1 compounds and one or more vitamin C compounds selected from the group consisting of vitamin C and the vitamin C derivatives, wherein the one or more vitamin C derivatives have the formula:

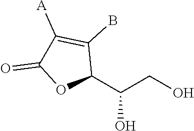

wherein each of A and B are independently OH, F, or methoxy, with the proviso that A and B are different from one another; and wherein there is a non-covalent association between the one or more vitamin B1 compounds and the one or more vitamin C compounds.

In certain embodiments, the Soxhlet extraction as described herein can be conducted using a solvent selected from the group consisting of ethanol, methanol, toluene, hexane, and combinations thereof.

In another embodiment, the non-covalent association obtained according to the present methods can be hydrogen bonding.

In a further embodiment, the one or more vitamin C derivatives can be 2-O-methylascorbic acid, 3-O-methylascorbic acid or a combination thereof synthesized using D-glucose as a starting material. The specific synthetic schemes for producing these vitamin C derivatives may involve protecting other hydroxyl groups of D-glucose using appropriate protecting groups, followed by reaction with an appropriate methylating agent, such as methyl iodide or methyl triflate, to introduce the O-methyl group at the desired position. Subsequent deprotection of other hydroxyl groups can yield the respective Vitamin C derivatives.

In an embodiment, during the production method, the one or more vitamin C derivatives can be positioned such that the A and B substituents are in close proximity to reactive sites of the one or more vitamin B1 compounds.

In this regard, optimal positioning of the derivatives within the complex, such as positioning the substituents (for example, F or OMe) in close proximity to the reactive sites of vitamin B1, can facilitate efficient electron transfer and stabilization of the radical intermediates.

For derivatives with F substitutions, positioning the F group in close proximity to the reactive sites of vitamin B1, such as the NI′ atom, could potentially enhance the antioxidant activity by promoting electron transfer and stabilization of the radical intermediates. Similarly, for derivatives with OMe substitutions, optimal positioning of the OMe group in close proximity to the reactive sites of vitamin B1, such as the pyrimidine ring or the thiazole ring, could potentially promote efficient electron transfer and stabilization of the radical intermediates, thereby enhancing the antioxidant activity.

The subject matter of the present application can be further understood by referring to the following examples.

### EXAMPLES

### Example 1

### Sample Preparation

Collected samples from myrrha resin were dried and then grounded to their fine form.

### Solvent Extraction Method

Vitamin samples were extracted by solvent extraction technique using Soxhlet (Ethanol, Methanol, Toluene and Hexane). About 1 g of Myrrh resin powder was weighed accurately and placed into the thimble in the Soxhlet chamber 100 ml of selected solvents were placed in a round bottom flask and assembled for Soxhlet extractor, then the distillation process was begun after completing the extraction. The final extract was filtered and placed into a polyethene container.

This method was repeated for the four different solvents identified above.

### Example 2

Eight samples of vitamins B1 and C were analyzed using High Liquid performance Chromatography, (Prominence-i (LC-2030, LIQUID CHROMATOGRAPHY) from SHIMADZU with column C18 100 mm, UV detector and RF 20 detector, Sample injector A 1.5 mL and B 1.5 mL, Mobil phase containers, normally A: Water and B: Methanol, 440 bar LC-2030 pump, Dual variable wavelength detector, Heater/Cooler oven and lab software solution consist of LC-2030 Controller, LC-2030 Auto sampler and LC-2030 Auto purge.

The prepared samples as mentioned above were diluted in 10 ml sodium hydrogen phosphate buffer (NaHPO4) pH 3.5 and filtered over a 0.4 μm pore size syringe filter.

### Mobile Phase

The mobile phase was prepared and consisted of 95%-NaHPO4 (0.025M) pH 4.5 and 5% Methanol (HPLC grade quality).

### Injection of Standard and Samples

10 μL of each sample was injected. The samples were separated at 30° C. at a flow rate of 1 mL/min with a linear gradient at wavelength 270 nm (San José Rodriguez et al., 2012).

### Standards Preparation

Stock standard solutions of Ascorbic Acid (Vitamin C) and Thiamine (Vitamin B1) (1000 μg/mL) were prepared by dissolving an appropriate amount directly in water and stored by keeping them protected from light and air at room temperature for one week. Working standard solutions were prepared daily.

### Example 3

2-O-methylascorbic acid and 3-O-methylascorbic acid can be synthesized from D-glucose as a starting material. The specific synthetic schemes may involve protecting other hydroxyl groups of D-glucose using appropriate protecting groups, followed by reaction with an appropriate methylating agent, such as methyl iodide or methyl triflate, to introduce the O-methyl group at the desired position. Subsequent deprotection of other hydroxyl groups yields the respective Vitamin C derivatives.

### Example 4

### BDE Analysis for Vitamin C

Bond Dissociation Energies (BDEs) of vitamin C and vitamin B1 were calculated and compared to the corresponding non-covalent complex. The weakest BDE value indicates the stronger antioxidant activity. The non-covalent interaction strongly reduced the BDE values, highly influenced by intermolecular hydrogen bonding, which significantly weakens the O—H bond. The general trend of the calculated BDEs indicates a slight decrease induced by the p-stacking interaction, which indicates the positive effect of that interaction on the antioxidant activity of the studied single vitamins.

### Example 5

### BDE Analysis for Vitamin C Derivatives

Table 1 below provides calculated values of bond dissociation enthalpies (BDEs) for vitamin C (ascorbic acid) derivatives with F and OMe substitutions at positions 2 and 3, both as single molecules and within a non-covalent complex with vitamin B1. These positions were selected because they are known to be critical for the antioxidant activity of ascorbic acid. The BDEs are important indicators of antioxidant activity, as lower BDE values generally correspond to higher antioxidant potential.

TABLE 1
Calculated Bond Dissociation Enthalpy (BDE) in kcal/mol.

| | | Single | |
|---|---|---|---|
| Derivative | Position | Molecule | Complex |
| Vitamin C (Ascorbic Acid) | 2 | 75 | 72 |
| Vitamin C (Ascorbic Acid) | 3 | 73 | 70 |
| 2-F-ascorbic acid | 2 | 71 | 69 |
| 3-F-ascorbic acid | 3 | 68 | 64 |
| 2-OMe-ascorbic acid | 2 | 69 | 67 |
| 3-OMe-ascorbic acid | 3 | 72 | 70 |

As it can be seen from Table 1, the BDE values within the non-covalent complex with vitamin B1 are found to be slightly lower than those of the corresponding single molecules for most derivatives. This suggests that the non-covalent association with vitamin B1 further enhances the antioxidant activity of these derivatives, as lower BDEs indicate higher antioxidant potential. The geometrical arrangement of the single molecules within the non-covalent complex of ascorbic acid derivatives with vitamin B1 also plays a crucial role in facilitating this synergistic effect and enhancing antioxidant activity (See FIGS. 1A-1C).

It is to be understood that the compositions, systems, and methods as described herein are not limited to the specific embodiments described above, but encompass any and all embodiments within the scope of the generic language of the following claims enabled by the embodiments described herein, or otherwise shown in the drawings or described above in terms sufficient to enable one of ordinary skill in the art to make and use the claimed subject matter.

1. A composition comprising:
vitamin B1; and
one or more vitamin C compounds selected from the group consisting of vitamin C and vitamin C derivatives,
wherein the vitamin C derivatives have the formula: 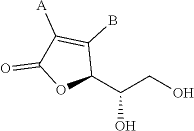
wherein the vitamin B1 and the vitamin C are extracted from Commiphora myrrha resin,
wherein each of A and B are independently OH, F, or methoxy, with the proviso that A and B are different from one another;
wherein the one or more vitamin C compounds include at least one of 2-O-methylascorbic acid and 3-O-methylascorbic acid, and wherein the 2-O-methylascorbic acid and 3-O-methylascorbic acid are synthesized from D-glucose as a starting material; and
wherein there is a non-covalent association between the vitamin B1 and the one or more vitamin C compounds, and wherein the vitamin B1 and the one or more vitamin C compounds are each present in the composition such that the composition exhibits a synergistic antioxidant effect due to the non-covalent association based on inclusion of both the vitamin B1 and the one or more vitamin C compounds.

2. The composition of claim 1, wherein the non-covalent association is hydrogen bonding.

3. (canceled)

4. The composition of claim 1, wherein the vitamin C derivatives are one or more of the following compounds 1-4:

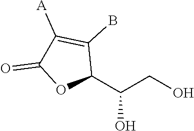

| | A | B |
|---|---|---|
| 1 | OH | F |
| 2 | F | OH |
| 3 | OH | Methoxy |
| 4 | Methoxy | OH |

5. (canceled)

6. A method of treating an oxidant condition in a subject, comprising:
administering a therapeutically effective amount of the composition of claim 1 to the subject in need thereof.

7. The method of claim 6, wherein the vitamin B1 and the one or more vitamin C compounds in the composition exhibit a synergistic antioxidant effect due to the non-covalent association.

8. The method of claim 7, wherein the non-covalent association is hydrogen bonding.

9. The method of claim 7, wherein the non-covalent association enhances antioxidant activity of the vitamin C derivatives.

10. A method of producing an antioxidant composition, the method comprising:
drying a sample of Commiphora myrrha Resin to obtain a dried resin;
grinding the dried resin to obtain a fine powder;
extracting a vitamin sample from the fine powder using Soxhlet extraction to obtain an extract;
filtering the extract to obtain a filtered composition; and
adding one or more vitamin C derivatives to the filtered composition to obtain the antioxidant composition containing one or more vitamin B1 compounds and one or more vitamin C compounds selected from the group consisting of vitamin C and the vitamin C derivatives,
wherein the one or more vitamin C derivatives have the formula: 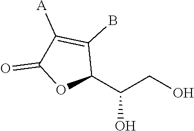
wherein each of A and B are independently OH, F, or methoxy, with the proviso that A and B are different from one another; and
wherein there is a non-covalent association between the one or more vitamin B1 compounds and the one or more vitamin C compounds.

11. The method of claim 10, wherein the Soxhlet extraction is conducted using a solvent selected from the group consisting of ethanol, methanol, toluene, hexane, and combinations thereof.

12. The method of claim 10, wherein the non-covalent association is hydrogen bonding.

13. The method of claim 10, wherein the one or more vitamin C derivatives are 2-O-methylascorbic acid, 3-O-methylascorbic acid or a combination thereof synthesized using D-glucose as a starting material.

14. The method of claim 10, wherein the one or more vitamin C derivatives are positioned such that the A and B substituents are in close proximity to reactive sites of the one or more vitamin B1 compounds.
